(19) United States
(12) Patent Application Publication
(10) Pub. No.: US 2022/0356500 A1
(43) Pub. Date: Nov. 10, 2022

(54) METHOD FOR PREPARING NUCLEIC ACID SEQUENCES USING ENZYME

(71) Applicant: YUANDIAN BIOLABS CO., LTD., Hsinchu (TW)

(72) Inventors: Cheng-Yao Chen, Hsinchu (TW); Jui-Kang Yen, Hsinchu (TW)

(21) Appl. No.: 17/789,533

(22) PCT Filed: Dec. 30, 2019
(86) PCT No.: PCT/US2019/068865

§ 371 Date: Jun. 28, 2022

(51) Int. Cl. C12P 19/34; C12N 9/12

(52) U.S. Cl. CPC C12P 19/34; C12N 9/1252

(57) ABSTRACT

A method for preparing nucleic acid sequences using an enzyme, including: (1) providing a reaction substrate having a pretreated surface. (2) Disposing a nucleotide having a terminal protecting group on the pretreated surface by a reaction enzyme, and a reaction temperature is 45° C.-105° C. (3) Removing the terminal protecting group of the nucleotide by irradiation or heating. (4) Coupling another nucleotide having the terminal protecting group to the nucleotide by the reaction enzyme, and a reaction temperature is 45° C.-105° C. (5) Determining whether nucleic acid sequence is completed, and if so, obtaining the nucleic acid sequence, if otherwise repeating steps (3) and (4). The method for preparing nucleic acid sequences using an enzyme of the invention may increase the efficiency of preparing nucleic acid sequences.

### FIELD OF THE INVENTION

The present invention relates to a method for preparing a biomolecular structure, and more particularly to a method for preparing nucleic acid sequences using an enzyme.

### BACKGROUND OF THE INVENTION

In the mid-20th century, there were several key breakthroughs in the fields of genetics and biochemistry that have led us to current day medicine. This was a cascade of events that started with X-ray-induced gene knockout studies in 1941, which made the connection that genes were directly involved in enzyme function. It soon followed that genes themselves were comprised of nucleic acids (DNA), and a double helix was an orderly structure of nucleic acids that stored genetic information and could be precisely replicated by a DNA polymerase.

Nucleic acid synthesis is vital to modern biotechnology. The rapid pace of development in the biotechnology arena has been made possible by the scientific community's ability to artificially synthesis DNA, RNA and proteins. Artificial DNA synthesis, a 1 billion and growing market, allows biotechnology and pharmaceutical companies to develop a range of peptide therapeutics, such as insulin for the treatment of diabetes. It allows researchers to characterize cellular proteins to develop new small molecule therapies for the treatment of diseases our aging population faces today, such as heart disease and cancer.

However, current DNA synthesis technology does not meet the demands of the biotechnology industry. While the benefits of DNA synthesis are numerous, an oft-mentioned problem prevents the further growth of the artificial DNA synthesis industry, and thus the biotechnology field. Despite being a mature technology, it is practically very hard to synthesis a DNA strand greater than 200 nucleotides in length, and most DNA synthesis companies only offer up to 120 nucleotides. In comparison, an average protein-coding gene is of the order of 2000-3000 nucleotides, and an average eukaryotic genome numbers in the billions of nucleotides. Thus, all major gene synthesis companies today rely on variations of a “synthesis and stitch” technique, where overlapping 40-60-mer fragments are synthesized and stitched together by PCR (see Young, L. et al. (2004) Nucleic Acid Res., 32, e59). Current methods offered by the gene synthesis industry generally allow up to 3 kb in length for routine production.

To date, there are two main categories of in vitro synthesis of nucleic acids: chemical syntheses or enzymatic syntheses. The most common in vitro nucleic acid chemical synthesis method is the phosphoramidite polymerization method described by Adams et al. (1983, J. Amer, Chem Soc., 105: 661) and Froehler et al. (1983, Tetrahedron Lett, 24: 3171). In this method, each nudeotide to be added is protected at the level of the 5′-OH group so as to avoid an uncontrolled polymerization of several nucleotides of the same type. Generally the protection of the 5′-OH group is carried out by a trityl group. In order to avoid possible degradation due to the use of powerful reagents, the bases carried by the nucleotides can also be protected. Generally the protection used involves an isobutyryl group (Reddy et al., 1997, Nucleosides & Nucleotides, 16: 1589). After each incorporation of new nucleotides, the 5′-OH group of the last nucleotide of the chain undergoes a deblocking reaction in order to make it available for the next polymerization step. The nitrogenous bases carried by the nucleotides composing the nucleic acid, they are deblocked only after completion of the complete polymerization.

WO 95/00530 A1 discloses a method for making oligonucleotide arrays by synthesizing oligonucleotide probes in situ on a substrate using photolithography. The oligonucleotides are immobilized on the substrate and synthesized base-wise in the 3′ to 5′ direction using light-sensitive protecting groups on the 5′ terminal hydroxyl groups. During each synthesis cycle, the protecting groups are selectively removed by illuminating the surface through a photolithographic mask; the deblocked hydroxyl groups are coupled to a selected 5′-photo-protected deoxynucleoside phosphoramidite, while the growing strands in the un-illuminated regions of the surface remain protected and cannot react. Rounds of illumination and coupling are repeated with different activated deoxynucleosides as required until the desired set of oligonucleotide probes is obtained.

US 20160184788A1 discloses a method of selectively masking one or more sites on a surface and a method of synthesizing an array of molecules. It immobilizes the nucleotides on the substrate and synthesizes base-wise in the 3′ to 5′ direction using thermo-sensitive protecting groups on the 5′ terminal hydroxyl groups. Thermo-sensitive protecting groups of nucleotides located at the sites can be removed by heating at different sites. The deblocked hydroxyl group is coupled to the selected 5′-photo-protected deoxynucleoside phosphoramidite, therefore a large number of different nucleic acid sequences can be prepared.

However, chemical synthesis methods, such as those discussed above, require large amounts of unstable, hazardous, expensive reagents that can impact the environment and health. The devices making it possible to carry out these syntheses in a practical way are complex, require a large investment and must be operated by a qualified and dedicated workforce. One of the major disadvantages of these chemical synthesis techniques lies in their low yield. During each cycle, the coupling reaction occurs only in 98 to 99.5% of the cases, leaving in the reaction medium nucleic acids that do not have a correct sequence. As the synthesis progresses, the reaction medium is greatly enriched in fragments with a totally incorrect sequence. The deletion type errors that occur thus have particularly dramatic repercussions causing a shift in the reading frame of the nucleic acid fragments considered.

Thus, for a correct coupling reaction in 99% of cases, a nucleic acid comprising 70 nucleotides will be synthesized with a yield of less than 50%. Which means that after 70 cycles of addition, the reaction medium will comprise more than fragments with a wrong sequence than fragments with a correct sequence. This mixture is then unsuitable for further use.

The methods of chemical synthesis of nucleic acid are therefore ineffective for the synthesis of long fragments because they generate a very large amount of fragments having an incorrect sequence, then considered as impurities. In practice, the maximum length of fragments that can be efficiently produced by these methods is between 50 and 100 nucleotides.

On the other hand, the difference between the methods of enzyme synthesis and chemical synthesis is that the method of enzyme synthesis uses an enzymatic catalyst for carrying out the coupling step. DNA polymerases are often used to synthesize DNA. DNA polymerases have been categorized in seven evolutionary families based on their amino acid sequences: A, B, C, D, X, Y, and RT. The families of DNA polymerases appear to be unrelated, i.e., members of one family are not homologous to members of any other family. A DNA polymerase is determined to be a member of given family by its homology to a prototypical member of that family. For example, members of family A are homologous to E. coli DNA polymerase I; members of family B are homologous to E. coli DNA polymerase II; members of family C are homologous to E. coli DNA polymerase III; members of family D are homologous to Pyrococcus furiosus DNA polymerase; members of family X are homologous to eukaryotic DNA polymerase beta; members of family Y are homologous to eukaryotic RAD30; and members of family RT are homologous to reverse transcriptase.

Various attempts have been made to use a terminal deoxynucleotidyl transferase for controlled de novo single-stranded DNA synthesis (Ud-Dean et al. (2009) Syst. Synth. Boil., 2, 67-73, U.S. Pat. Nos. 5,763,594 and 8,808,989). Uncontrolled de novo single-stranded DNA synthesis, as opposed to controlled, takes advantage of TdT's deoxynucleotide triphosphate (dNTP) 3′ tailing properties on single-stranded DNA to create, for example, homopolymeric adaptor sequences for next-generation sequencing library preparation (Roychoudhur R. et al. (1976) Nucleic Acids Res. 3, 10-116 and WO 2003/050242).

However, as its key purpose is to increase antigen receptor diversity, terminal deoxynucleotidyl transferase may resist normal, predictable behavior observed with most replicative, high-fidelity polymerases. These represent many challenges to its use in the nucleotide synthesis cycle for high-throughput automation.

In summary, the chemical synthesis of nucleic acids can synthesize nucleic acids in large quantities. However, the reagents used are too expensive and pollute the environment, and the probability of error in the synthesis process is higher. The enzymatic synthesis of nucleic acids is inexpensive, and the nucleic acid sequences have a higher degree of accuracy. However, due to the reaction rate of the enzyme, large quantities of synthesis cannot be performed. Therefore, there is still no better way to synthesize nucleic acids today, which can solve the problem of correct and large quantities of synthesis of nucleic acids.

### SUMMARY OF THE INVENTION

The present invention provides a method for preparing nucleic acid sequences using an enzyme, which increases the efficiency of preparing nucleic acid sequences.

A method for preparing nucleic acid sequences using an enzyme provided in an embodiment of the invention includes: (1) providing a reaction substrate having a pretreated surface. (2) Disposing a nucleotide having a terminal protecting group on the pretreated surface by a reaction enzyme, and a reaction temperature is 45° C.-105° C. (3) Removing the terminal protecting group of the nucleotide by irradiation or heating. (4) Coupling another nucleotide having the terminal protecting group to the nucleotide by the reaction enzyme, and a reaction temperature is 45° C.-105° C. (5) Determining whether a nucleic acid sequence is completed, and if so, obtaining the nucleic acid sequence, if otherwise repeating steps (3) and (4).

In one embodiment of the invention, the reaction enzyme is a DNA polymerase.

In one embodiment of the invention, the reaction enzyme is family A DNA polymerase.

In one embodiment of the invention, the reaction enzyme is family B DNA polymerase.

In one embodiment of the invention, the reaction enzyme is family X DNA polymerase.

In one embodiment of the invention, the method of removing the terminal protecting group of the nucleotide by irradiation or heating includes partially removing in a patterned manner.

In one embodiment of the invention, the method of removing the terminal protecting group of the nucleotide by irradiation includes using a UV digital light processing (DLP) chip for irradiation.

In one embodiment of the invention, the reaction temperature is 50° C.-85° C.

In one embodiment of the invention, the reaction temperature is 55° C.-75° C.

In one embodiment of the invention, the pretreated surface has a plurality of primers, in which the method of disposing a nucleotide having a terminal protecting group on the pretreated surface includes coupling the nucleotide having the terminal protecting group to the primers by the reaction enzyme.

In one embodiment of the invention, the method for preparing nucleic acid sequences using an enzyme further including: (6) cutting the nucleic acid sequence from the pretreated surface by a restriction enzyme.

Since the method for preparing nucleic acid sequences using an enzyme of the embodiment of the invention use an enzyme to synthesize a nucleic acid sequence, it is less likely to pollute the environment and may reduce the cost compared to the method of chemical synthesis. In addition, the reactor has an operating temperature of 45° C.-105° C., compared to the conventional use of enzyme at 37° C., the embodiment of the invention may exhibit better activity by using an enzyme at 45° C. or higher, thereby increasing the efficiency of preparing nucleic acid sequences.

Other objectives, features and advantages of The invention will be further understood from the further technological features disclosed by the embodiments of The invention wherein there are shown and described preferred embodiments of this invention, simply by way of illustration of modes best suited to carry out the invention.

### BRIEF DESCRIPTION OF THE DRAWINGS

The present invention will become more readily apparent to those ordinarily skilled in the art after reviewing the following detailed description and accompanying drawings, in which:

FIG. 1 is a schematic flow diagram of a method for preparing nucleic acid sequences using an enzyme of one embodiment of the invention;

FIG. 2A and FIG. 2B are schematic diagrams of removing a terminal protecting group of a nucleotide of one embodiment of the invention;

FIG. 3A and FIG. 3B are schematic diagrams of removing a terminal protecting group of a nucleotide of another embodiment of the invention;

FIG. 4A and FIG. 4B are schematic diagrams of removing a terminal protecting group of a nucleotide of another embodiment of the invention; and

FIG 5A to FIG. 5I are schematic diagrams of steps of a method for preparing nucleic acid sequences using an enzyme of one embodiment of the invention.

### DETAILED DESCRIPTION OF PREFERRED EMBODIMENTS

The present invention will now be described more specifically with reference to the following embodiments. It is to be noted that the following descriptions of preferred embodiments of this invention are presented herein for purpose of illustration and description only. It is not intended to be exhaustive or to be limited to the precise form disclosed.

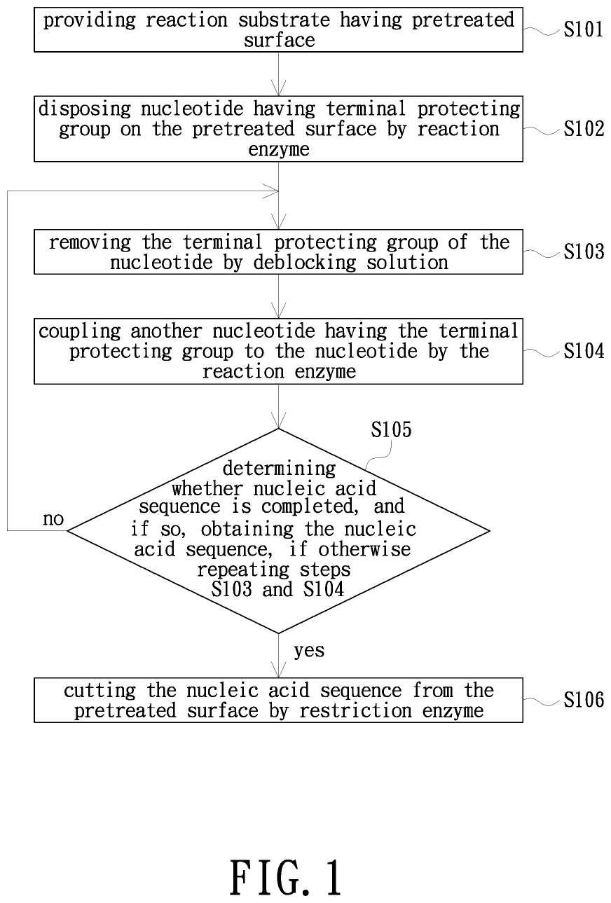

FIG. 1 is a schematic flow diagram of a method for preparing nucleic acid sequences using an enzyme of one embodiment of the invention. Referring to FIG. 1, a method for preparing nucleic acid sequences using an enzyme of the embodiment includes the following steps. Step S101: providing a reaction substrate having a pretreated surface. Next, step S102: disposing a nucleotide having a terminal protecting group on the pretreated surface by a reaction enzyme, and a reaction temperature is 45° C.-105° C., preferably 50° C.-85° C., more preferably 55° C.-75° C. Specifically, the reaction temperature for preparing the nucleic acid sequences is, for example, 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C. or 75° C., but is not limited thereto.

A material of the reaction substrate includes, for example, silicon, glass (SiO₂), a metal or a polymer such as polycarbonate or polymethyl methacrylate, but is not limited thereto. Specifically, the reaction substrate is, for example, a plate-like structure such as a wafer or a plate, and the pretreated surface is a plane of the plate-like structure.

In the embodiment, a synthesis of deoxyribonucleic acid (DNA) sequences is taken as an example. The nitrogenous bases of the nucleotides required for their synthesis may be further divided into four categories: adenine (A), thymine (T), cytosine (C), guano (G). Therefore, four different types of nucleotides (dAMP, dTMP, dCMP, dGMP) may be used. When synthesizing a ribonucleic acid (RNA) sequence, a uracil nucleotide (UMP) is also required. The reaction enzyme is, for example, a DNA polymerase, particularly a heat stable DNA polymerase, but is not limited thereto. The DNA polymerase includes, for example, family A DNA polymerase, family B DNA polymerase and family X DNA polymerase, and examples thereof include Taq DNA polymerase, archaeal DNA polymerase or thermally stable reverse transcriptase. These DNA polymerases may exhibit better activity at a temperature of 45° C. or higher than the terminal deoxynucleotidyl transferase (TdT) which is conventionally used at 37° C., thereby improving the synthesis efficiency of the nucleic acid sequences.

As used herein, the terms “reaction enzyme”, “nucleotide having terminal protecting group”, “nucleotide”, “restriction enzyme” and the like described throughout the present invention shall be regarded as a general term for these substances, not the actual quantity thereof. For example, the quantity of the reaction enzyme for reaction is plural, and the quantity of the nucleotide having the terminal protecting group is also plural.

The method of disposing the nucleotide having the terminal protecting group on the pretreated surface by the reaction enzyme includes, for example, immersing method or liquid delivering method. Specifically, the reaction enzyme and the nucleotide having the terminal protecting group are prepared into a formulation solution. In immersing method, the reaction substrate is immersed in the formulation solution, and the temperature of the solution is maintained at 45° C.-105° C., waiting for the reaction to complete. In liquid delivering method, the formulation solution is passed over the pretreated surface of the reaction substrate, and the temperature of the reaction substrate is maintained at 45° C.-105° C. The methods are only the embodiments of the invention and are not intended to limit the scope of the invention.

The pretreated surface has, for example, a plurality of primers, but is not limited thereto. The method of disposing the nucleotide having the terminal protecting group on the pretreated surface includes coupling the nucleotide having the terminal protecting group to the primers by the reaction enzyme. In the embodiment, when the DNA sequences are synthesized, the DNA polymerase (reaction enzyme) may more easily dispose the nucleotide having the terminal protecting group on the pretreated surface by the help of the primers. These primers are, for example, single-stranded DNA, but are not limited thereto. In another embodiment, the pretreated surface may, for example, have no primers.

Next, step S103: removing the terminal protecting group of the nucleotide by irradiation or heating. The terminal protecting group is, for example, photo-sensitive or thermo-sensitive. Examples of the terminal protecting group include methyl, 2-nitrobenzyl, 3′-O-(2-cyanoethyl), allyl, amine, azidomethyl, tert-butoxy ethoxy (TBE) and the like, but are not limited thereto. The photo-sensitive terminal protecting group may be removed by irradiation, while the thermo-sensitive terminal protecting group may be removed by heating. After the removal of the terminal protecting group, the nucleotide may continue to couple the next nucleotide to extend the sequence. The specific implementation of removing the terminal protecting group of the nucleotide will be further described below with reference to the drawings, but the specific method of removing the terminal protecting group of the nucleotide by irradiation or heating of the invention is not limited to the embodiments listed below.

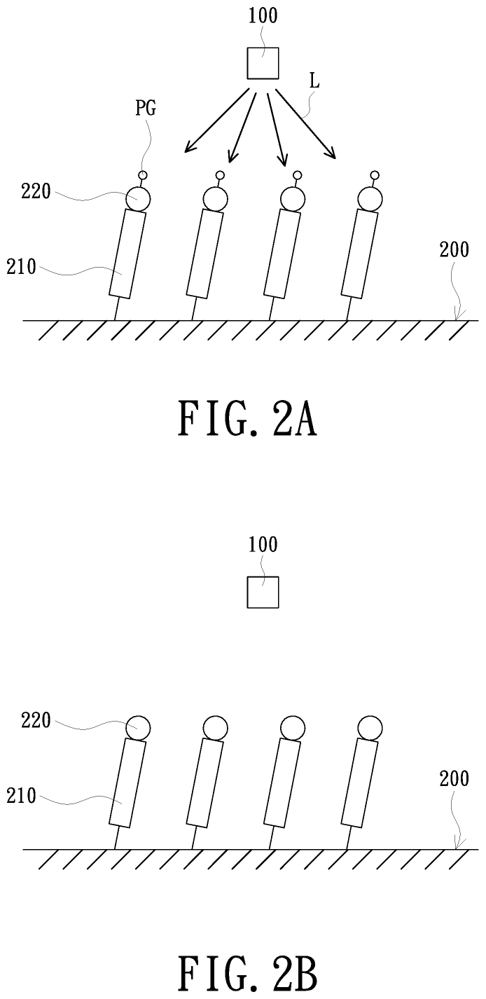

FIG. 2A and FIG. 2B are schematic diagrams of removing a terminal protecting group of a nucleotide of one embodiment of the invention. Referring to FIG. 2A and FIG. 2B, in the embodiment, a photo-sensitive terminal protecting group PG is used, and a pretreated surface 200 is irradiated by a light emitting element 100, so that the terminal protecting group PG of the nucleotide 220 coupled to a primer 210 is decomposed after being irradiated by a light beam L, as shown in FIG. 2B. The light emitting element 100 is, for example, a light emitting diode or a laser diode, but is not limited thereto. The irradiation is not limited to the use of the light emitting element 100, for example, natural light irradiation may also be used. Since a plurality of nucleotides 220 may be simultaneously disposed on the pretreated surface 200, a plurality of nucleic acid sequences may be prepared at one time, thereby improving the efficiency of preparing the plurality of nucleic acid sequences.

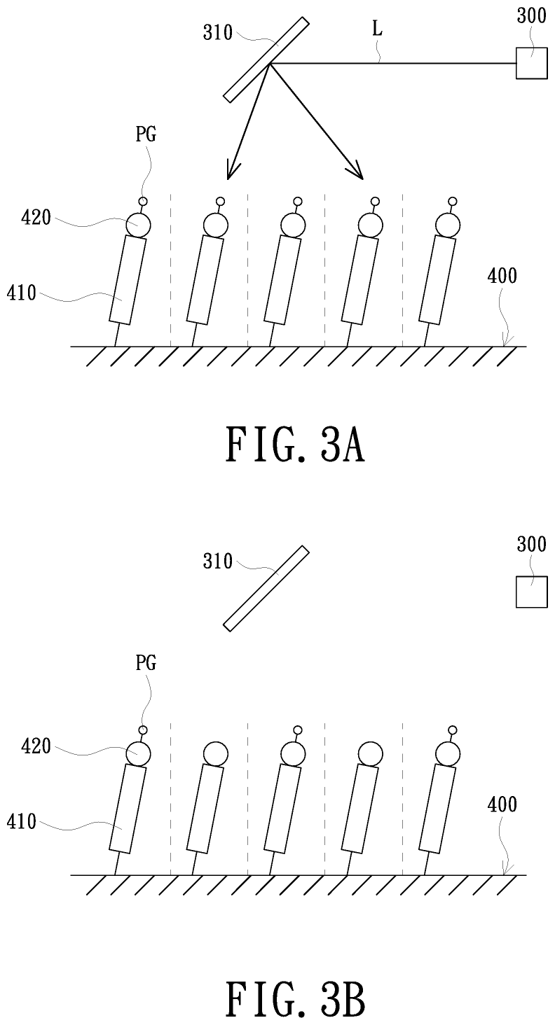

In addition, the pretreatment surface 200 may also be divided into a plurality of regions, each of which includes a nucleic acid sequence in preparation, and the terminal protecting group PG is partially removed in a patterned manner to achieve the effect of simultaneously preparing a plurality of different nucleic acid sequences. FIG. 3A and FIG. 3B are schematic diagrams of removing a terminal protecting group of a nucleotide of another embodiment of the invention. Referring to FIG. 3A and FIG. 3B, in the embodiment, the photo-sensitive terminal protecting group PG is also used, the difference being that the embodiment uses a UV digital light processing (DLP) chip for irradiation. Specifically, the UV digital light processing chip includes a light emitting element 300 and a reflective light valve 310. The plurality of regions in FIG. 3A and FIG. 3B are separated by dashed lines. After the light emitting element 300 emits a light beam L to the reflective light valve 310, partial of the plurality of regions of the pretreated surface 400 are irradiated by the reflective light valve 310 in a patterned manner, so that the terminal protecting group PG of the nucleotide 420 coupled to the primer 410 in these regions is decomposed after being irradiated by the light L, and the nucleotide 420 may be coupled to the next nucleotide 420, as shown in FIG. 3B. By designing a patterned manner, different nucleic acid sequences may be prepared simultaneously in each region.

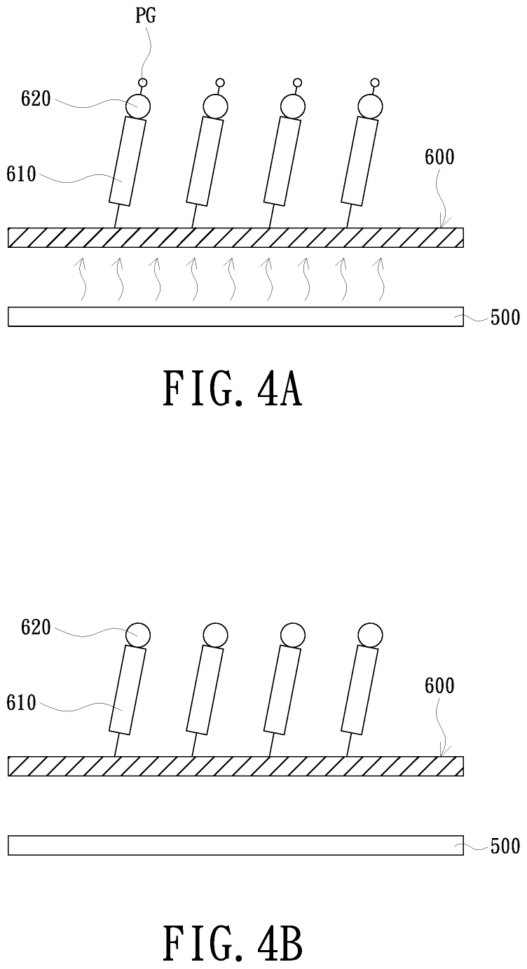

FIG. 4A and FIG. 4B are schematic diagrams of removing a terminal protecting group of a nucleotide of another embodiment of the invention. Referring to FIG. 4A and FIG. 4B, the method of the embodiment is similar to the above method, the difference is only that the embodiment uses a thermo-sensitive terminal protecting group PG Therefore, a pretreated surface 600 is heated by a heating element 500 (the thermal energy transfer is indicated by a curved arrow), and the terminal protecting group PG of the nucleotide 620 coupled to the primer 610 is decomposed after heating, as shown in FIG. 4B. The heating may, for example, also partially remove the terminal protecting group PG in a patterned manner, that is, in the way of regional heating. For example, when a silicon chip is used as a reaction substrate, a circuit may be distributed in each region to control a passing current to achieve the effect of regional heating.

Referring again to FIG. 1, after removing the terminal protecting group of the nucleotide, step S104 is performed: coupling another nucleotide having the terminal protecting group to the nucleotide disposed on the pretreated surface by the reaction enzyme. The reaction of step S104 is similar to step S102, except that the nucleotide having the terminal protecting group is coupled to the nucleotide which has been previously coupled to the primer. Depending on a length of the designed nucleic acid sequence, step S105 is followed: determining whether a nucleic acid sequence is completed, and if so, obtaining the nucleic acid sequence, if otherwise repeating steps S103 and S104 to continue extending the length of the nucleic acid sequence until the designed nucleic acid sequence is completed. If step S103 is to partially remove the terminal protecting group in different regions in a patterned manner when the steps are repeated, the lengths of the nucleic acid sequences in different regions may be different. The specific implementation of the method for preparing nucleic acid sequences using an enzyme will be further described below with reference to the drawings, but the specific method of the method for preparing nucleic acid sequences using an enzyme of the invention is not limited to the embodiments listed below.

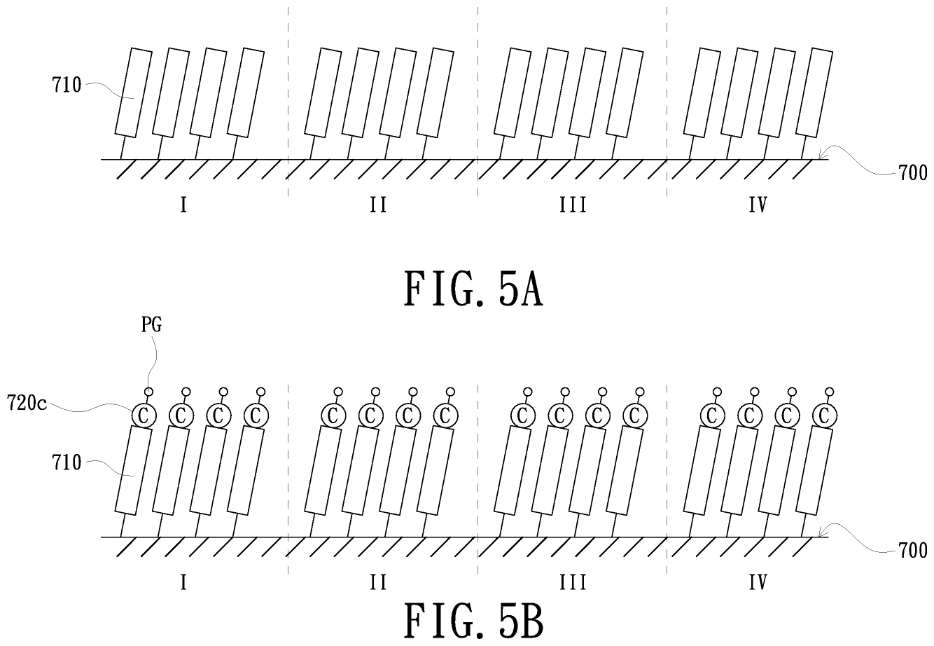

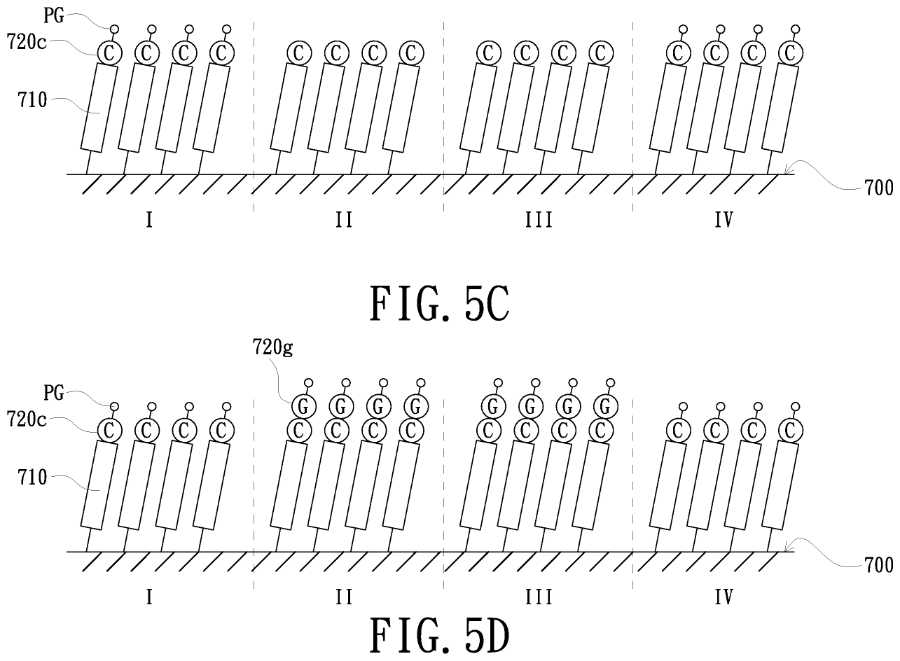

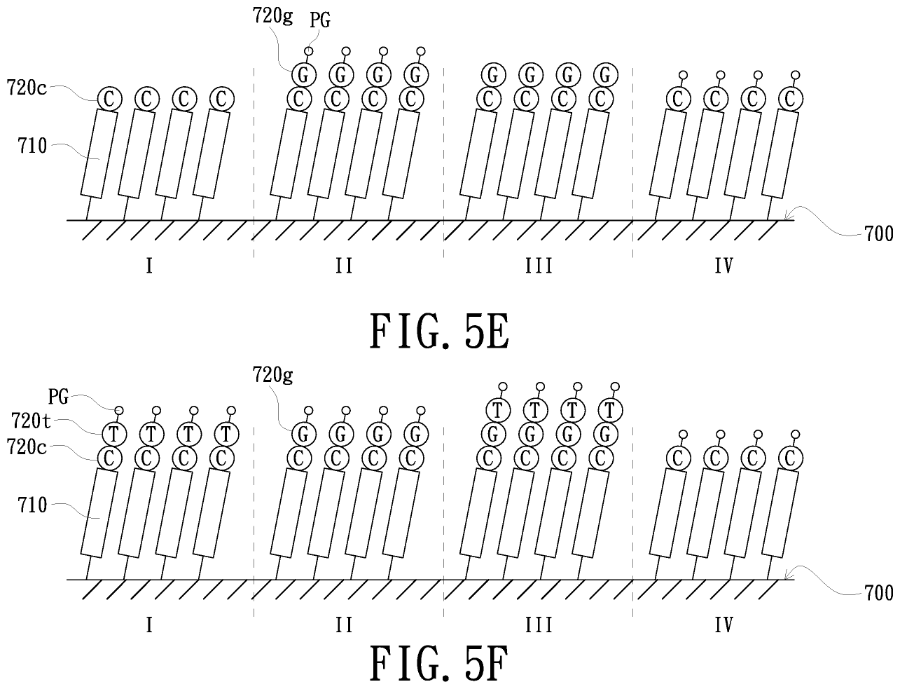

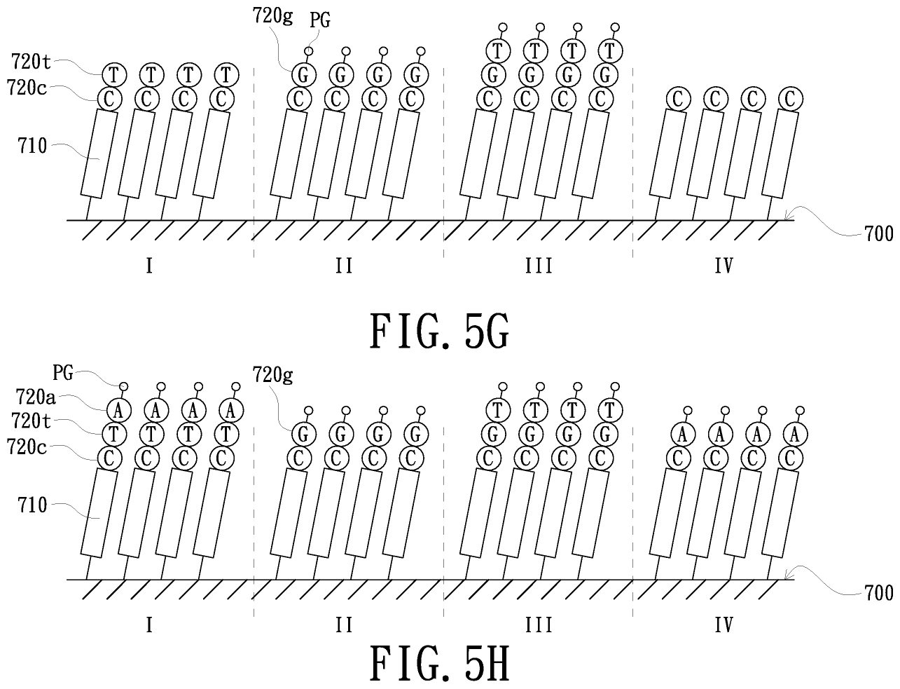

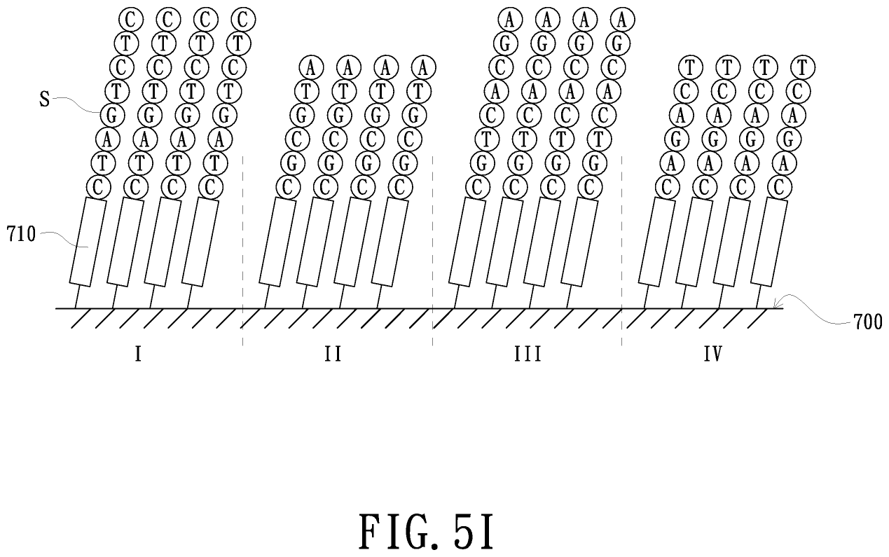

FIG. 5A to FIG. 5I are schematic diagrams of steps of a method for preparing nucleic acid sequences using an enzyme of one embodiment of the invention. Referring to FIG. 1, FIG. 5A to FIG 5I, step S101 is performed in FIG. 5A: providing a reaction substrate having a pretreated surface 700, the pretreated surface 700 has a plurality of primers 710 thereon. In the embodiment, the plurality of primers 710 are, for example, single-stranded DNA, but are not limited thereto. The pretreated surface 700 herein is divided into four regions: I, II, III and IV. Each region has a plurality of primers 710, and the regions I, II, III, and IV are designed to produce different nucleic acid sequences respectively.

Step S102 is performed in FIG. 5B: disposing a nucleotide 720c having a terminal protecting group PG on the pretreated surface 700 by a reaction enzyme, and a reaction temperature is 45° C.-105° C. The nucleotides 720 used in the embodiment include nucleotides 720a, 720c, 720g, 720t, which correspond to adenine nucleotide (dAMP), cytosine nucleotide (dCMP), guanine nucleotides (dGMP) and thymidine (dTMP), respectively. The nucleotides 720c disposed in FIG. 5B are cytosine nucleotides.

Step S103 is performed in FIG. 5C: removing the terminal protecting group PG of the nucleotide 720 by irradiation or heating. The terminal protecting groups PG in different regions is partially removed in a patterned manner and the embodiment of irradiation or heating has been described in detail and will not be repeated here. The terminal protecting groups PG of nucleotides 720c of the regions II, III are removed in FIG. 5C.

Step S104 is performed in FIG. 5D: coupling another nucleotide 720 having the terminal protecting group PG to the nucleotide 720 disposed on the pretreated surface 700 by the reaction enzyme. In FIG. 5D, only the nucleotides 720c of the regions II, III are additionally coupled to another nucleotide 720g by the reaction enzyme.

Next, step S105: determining whether a nucleic acid sequence is completed, and if so, obtaining the nucleic acid sequence, if otherwise repeating the step S103 and the step S104. Step S103 is performed again in FIG. 5E, the terminal protecting groups PG of the nucleotides 720c of the region I and the terminal protecting groups PG of the nucleotides 720g of the region III were removed. Next, Step S104 is performed again in FIG. 5F, another nucleotide 720t having the terminal protecting group PG is coupled to the nucleotides 720c of the region I and the nucleotides 720g of the region III by the reaction enzyme.

Then, step S105 is performed again to determine whether the nucleic acid sequence is completed, and if not completed, repeating the step S103 and the step S104. The terminal protecting groups PG of the nucleotides 720t of the region I and the terminal protecting groups PG of the nucleotides 720c of the region IV were removed in FIG. 5G. Another nucleotide 720a having the terminal protecting group PG is coupled to the nucleotides 720t of the region I and the nucleotides 720c of the region IV by the reaction enzyme in FIG. 5H.

The step S103 and the step S104 are repeated until the different nucleic acid sequences S in the regions I, II, III and IV are respectively synthesized, as shown in FIG. 5I. The above is the implementation aspect of the embodiment in which the terminal protecting groups PG in different regions I, II, III, and IV are partially removed by irradiation or heating in a patterned manner. By the method, a large quantity of different nucleic acid sequences S may be prepared in a short time. When the different nucleic acid sequences S are sequence fragments belonging to a same gene, by subsequent binding of the sequence fragments, a nucleic acid sequence having a longer length than that of the prior art may be produced in an amount.

After the synthesis of the nucleic acid sequences S is completed, the method for preparing nucleic acid sequences using an enzyme further includes, for example, step S106: cutting the nucleic acid sequences S from the pretreated surface 700 by a restriction enzyme. When the restriction enzyme is disposed on the pretreated surface 700, the restriction enzyme cuts the synthesized nucleic acid sequences S from the pretreated surface 700. The nucleic acid sequences S are then collected to complete the preparation process of the nucleic acid sequences S. Examples of the restriction enzyme include Uracil DNA Glycosylase (UDG) and Endonuclease VIII, or USER enzyme (NEB #M5508) and a combination thereof. For example, when a restriction enzyme that is cut to the uracil nucleotide (UMP) is used, before the reaction enzyme disposes the nucleotide 720 having the terminal protecting group PG to the primer 710, a uracil nucleotide is first disposed to the primer 710. When the nucleic acid sequences are completed, since they are DNA sequences, the DNA sequences may not contain the uracil nucleotide. For the restriction enzyme, only the previously disposed single uracil nucleotide may be cut, thereby being able to correctly cut the nucleic acid sequences S. The different restriction enzymes will have different cutting sites, and the invention is not particularly limited.

The pretreated surface surfaces 200, 400, 600, 700 use different numerical symbols only to distinguish different embodiments, and may be interchanged with each other for use in various embodiments. The primers 210, 410, 610, 710 and the nucleotides 220, 420, 620, 720a, 720c, 720g, 720t are also used in the same manner.

In summary, since the method for preparing nucleic acid sequences using an enzyme of the embodiment of the invention use an enzyme to synthesize a nucleic acid sequence, it is less likely to pollute the environment and may reduce the cost compared to the method of chemical synthesis. In addition, the reactor has an operating temperature of 45° C.-105° C., compared to the conventional use of enzyme at 37° C., the embodiment of the invention may exhibit better activity by using an enzyme at 45° C. or higher, thereby increasing the efficiency of preparing nucleic acid sequences.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A method for preparing nucleic acid sequences using an enzyme, comprising:
(1) providing a reaction substrate having a pretreated surface;
(2) disposing a nucleotide having a terminal protecting group on the pretreated surface by a reaction enzyme, and a reaction temperature is 45° C.-105° C.;
(3) removing the terminal protecting group of the nucleotide by irradiation or heating;
(4) coupling another nucleotide having the terminal protecting group to the nucleotide by the reaction enzyme, and a reaction temperature is 45° C.-105° C.; and
(5) determining whether a nucleic acid sequence is completed, and if so, obtaining the nucleic acid sequence, if otherwise repeating steps (3) and (4).

2. The method for preparing nucleic acid sequences using an enzyme according to claim 1, wherein the reaction enzyme is a DNA polymerase.

3. The method for preparing nucleic acid sequences using an enzyme according to claim 2, wherein the reaction enzyme is family A DNA polymerase.

4. The method for preparing nucleic acid sequences using an enzyme according to claim 2, wherein the reaction enzyme is family B DNA polymerase.

5. The method for preparing nucleic acid sequences using an enzyme according to claim 2, wherein the reaction enzyme is family X DNA polymerase.

6. The method for preparing nucleic acid sequences using an enzyme according to claim 1, wherein the method of removing the terminal protecting group of the nucleotide by irradiation or heating comprises partially removing in a patterned manner.

7. The method for preparing nucleic acid sequences using an enzyme according to claim 1, wherein the method of removing the terminal protecting group of the nucleotide by irradiation comprises using a UV digital light processing (DLP) chip for irradiation.

8. The method for preparing nucleic acid sequences using an enzyme according to claim 1, wherein the reaction temperature is 50° C.-85° C.

9. The method for preparing nucleic acid sequences using an enzyme according to claim 1, wherein the reaction temperature is 55° C.-75° C.

10. The method for preparing nucleic acid sequences using an enzyme according to claim 1, wherein the pretreated surface has a plurality of primers, in which the method of disposing a nucleotide having a terminal protecting group on the pretreated surface comprises coupling the nucleotide having the terminal protecting group to the primers by the reaction enzyme.

11. The method for preparing nucleic acid sequences using an enzyme according to claim 1, further comprising: (6) cutting the nucleic acid sequence from the pretreated surface by a restriction enzyme.
